(19) United States
(12) Patent Application Publication
(10) Pub. No.: US 2007/0282254 A1
(43) Pub. Date: Dec. 6, 2007

(54) Needle devices and methods

(76) Inventor: Mina Chow, Cambell, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/435,417

(22) Filed: May 16, 2006

(51) Int. Cl. A61M 29/00

(52) U.S. Cl. 604/96.01

(57) ABSTRACT

Methods and delivery devices for maximizing injectate dispersion in lesioned tissue using needle-based injection devices are herein disclosed. The delivery devices include injection devices with modified needle tip configurations. The needle tip configurations can include multiple circumferential openings.

### FIELD OF INVENTION

Modified needle tips.

### BACKGROUND OF INVENTION

Percutaneous transluminal coronary angioplasty (PTCA) is a procedure for treating heart disease, particularly, stenosis. “Stenosis” refers to a narrowing or constriction of the diameter of a vessel. In a typical PTCA procedure, a catheter assembly having a balloon portion is introduced percutaneously into the cardiovascular system of a patient via the brachial or femoral artery to treat stenosis at a lesion site. The catheter assembly is advanced through the coronary vasculature until the balloon portion is positioned across the occlusive lesion. Once in position across the lesion, the balloon is inflated to a predetermined size to radially compress the atherosclerotic plaque of the lesion against the inner wall of the artery to dilate the lumen. The balloon is then deflated to a smaller profile to allow the catheter to be withdrawn from the patient's vasculature.

Restenosis of the artery commonly develops over several months after the procedure, which may require another angioplasty procedure or a surgical by-pass operation. “Restenosis” is the reoccurrence of stenosis in a blood vessel or heart valve after it has been treated with apparent success. Restenosis is thought to involve the body's natural healing process. Angioplasty or other vascular procedures often injure the vessel walls, including removing the vascular endothelium, disturbing the tunica intima, and causing the death of medial smooth muscle cells. Excessive neoinitimal tissue formation, characterized by smooth muscle cell migration and proliferation to the intima, follows the injury. Proliferation and migration of smooth muscle cells (SMC) from the media layer to the intima cause an excessive production of extra cellular matrices (ECM), which is believed to be one of the leading contributors to the development of restenosis. The extensive thickening of the tissues narrows the lumen of the blood vessel, constricting or blocking blood flow through the vessel.

To reduce the chance of the development of restenosis, treatment substances can be administered to the treatment site. For example, anticoagulant and antiplatelet agents are commonly used to inhibit the development of restenosis. In order to provide an efficacious concentration to the target site, systemic administration of such medication often produces adverse or toxic side effects for the patient. Local delivery is a preferred method of treatment in that smaller total levels of medication are administered in comparison to systemic dosages, but are concentrated at a specific site. Local delivery, thus, produces fewer side effects and achieves more effective results.

Techniques for the local delivery of a treatment substance into the tissue surrounding a vessel are disclosed in U.S. Pat. Nos. 6,944,490, 6,692,466 and 6,554,801 to Chow et al. In some applications, such techniques include a catheter with a needle cannula slidably disposed in a needle lumen and a balloon, which is coupled to the distal end of the catheter. When the balloon is inflated the needle lumen is brought into close engagement with the tissue and the needle cannula can be moved between a position inboard of the catheter distal surface and a position where the needle cannula is projected outboard of the catheter to deliver the treatment substance to the tissue.

Needles which are used in conjunction with percutaneous injection devices and open-chest surgical injection devices generally include beveled single-port needle tips. Some of the problems associated with these types of needle tips include backflow of the injectate to non-focal areas, damage to surrounding tissue due to high focal injection pressure and reduced treatment agent dispersion due to localized delivery from a single port. Some studies have shown that up to 90 percent of the injectate never reaches the target tissue area due to backflow. As a result, treatment using needles often requires multiple injections which can result in increased pain and risk to the patient in addition to increased tissue damage due to multiple puncture wounds.

The treatment of organs with injection devices, in particular dynamic organs, also presents unique challenges. For example, the heart will generally be contracting during a treatment which increases backflow during each muscle contraction and decreases treatment agent dispersion.

### SUMMARY OF INVENTION

Methods and delivery devices for maximizing injectate dispersion in lesioned tissue using needle-based injection devices are herein disclosed. The delivery devices can be modified needle tip configurations. The needle tip configurations can include circumferential openings recesses, grooves and/or indentations.

### BRIEF DESCRIPTION OF DRAWINGS

FIGS. 1A-1C illustrate a substance delivery assembly which may be used in conjunction with embodiments of the present invention.

FIG. 2A illustrates an alternative delivery assembly which may be used in conjunction with embodiments of the present invention.

FIG. 2B illustrates a second alternative delivery assembly which may be used in conjunction with embodiments of the present invention.

FIG. 3 illustrates a delivery device of the prior art.

FIGS. 4A-4C illustrate embodiments of delivery devices of the present invention.

FIGS. 5A-5B illustrate alternative embodiments of delivery devices of the present invention.

FIG. 6 illustrates a another alternative embodiment of a delivery device of the present invention.

### DETAILED DESCRIPTION

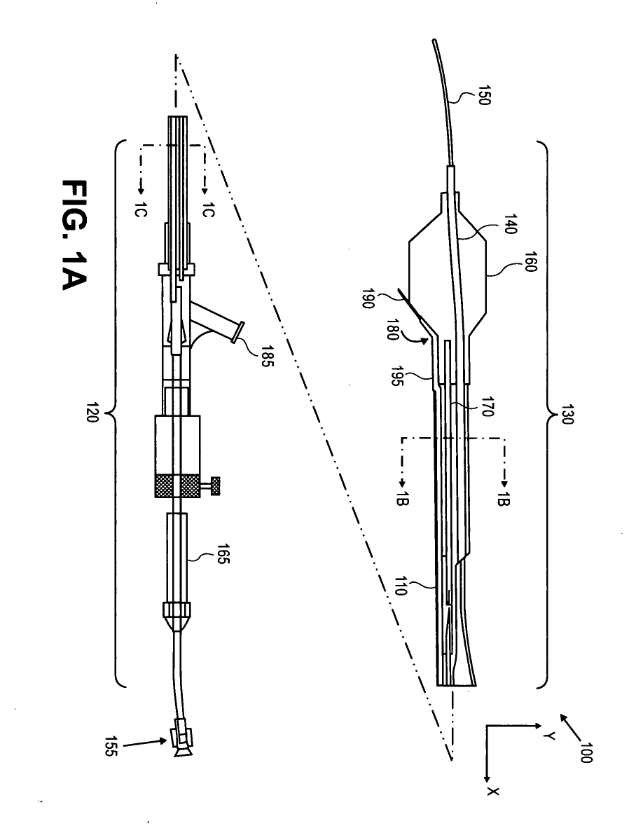

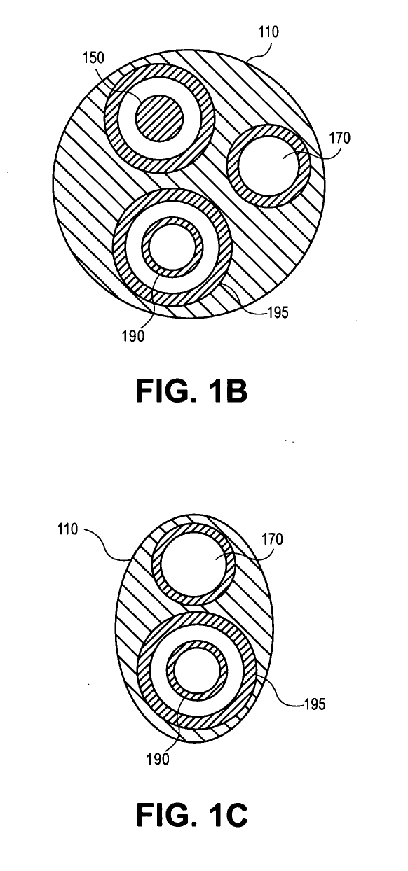

Methods and delivery devices for maximizing injectate dispersion in lesioned tissue using needle-based injection devices are herein disclosed. In some applications, the injection device, or delivery assembly hereinafter referred to interchangeably, may be a percutaneous injection device such as a balloon catheter assembly or a catheter assembly. In some applications, the injection device may be a hypodermic needle syringe. Representative injection devices are depicted in FIGS. 1-2B.

FIGS. 1A, 1B, and 1C illustrate a delivery assembly or device which can be used in conjunction with embodiments of the present invention. In general, the delivery assembly provides a system for delivering a substance, such as a treatment agent or a combination of treatment agent, to or through a desired area of a vessel in order to treat a localized area of the vessel or to treat a localized area of tissue located adjacent to the vessel. The delivery assembly includes a catheter assembly 100, which is intended to broadly include any medical device designed for insertion into a vessel to permit injection and/or withdrawal of fluids, to maintain the patency of the vessel, or for any other purpose. It is contemplated that the delivery assembly has applicability for use with any vessel or organ, including blood vessels, urinary tract, intestinal tract, kidney ducts, wind pipes, and the like.

In one embodiment, catheter assembly 100 is defined by an elongated catheter body 110 having a proximal end 120 and a distal end 130. Catheter assembly 100 can include a guidewire lumen 140 for allowing catheter assembly 100 to be fed and maneuvered over a guidewire 150. A balloon 160 is incorporated at distal end 130 of catheter assembly 100 and is in fluid communication with an inflation lumen 170 of catheter assembly 100.

Balloon 160 may be inflated by the introduction of a liquid into inflation lumen 170. Liquids containing treatment and/or diagnostic agents may also be used to inflate balloon 160. In one embodiment, balloon 160 may be made of a material that is permeable to such treatment and/or diagnostic liquids. To inflate balloon 160, the fluid can be supplied into inflation lumen 170 at a predetermined pressure, for example, between about 1 and 20 atmospheres. The specific pressure depends on various factors, such as the thickness of balloon wall, the material from which balloon wall is made, the type of substance employed, and the flow-rate that is desired.

Catheter assembly 100 also includes a substance delivery assembly 180 for injecting a substance into a wall of a vessel or tissue located adjacent to the vessel. In one embodiment, delivery assembly 180 includes a needle 190 movably disposed within a hollow delivery lumen 195. Needle 190 includes a lumen with an inside diameter of, representatively, about 0.08 inches (0.20 centimeters). Delivery lumen 195 extends between distal end 130 and proximal end 120. Delivery lumen 195 can be made from any suitable material, such as polymers and copolymers of polyamides, polyolefins, polyurethanes and the like. Access to the proximal end of delivery lumen 195 for insertion of needle 190 is provided through a hub 185.

Needle 190 is slidably or movably disposed in delivery lumen 195. Needle 190 includes a tissue-piercing tip having a dispensing port (not shown). The dispensing port is in fluid communication with a central lumen (not shown) of needle 190. In one embodiment, the central lumen of needle 190 can be pre-filled with a measured amount of a substance. The central lumen of needle 190 connects the dispensing port with substance injection port 155, which is configured to be coupled to various substance dispensing means of the type well known in the art, such as, for example, a syringe or fluid pump. Injection port 155 allows a measured substance to be dispensed from a dispensing port as desired or on command. In some applications, catheter assembly 100 enters percutaneously through an arterial vessel of the heart.

FIG. 2A illustrates a cross-sectional side view of an alternative delivery device or apparatus which can be used in conjunction with embodiments of the present invention. In general, delivery assembly 200 provides an apparatus for delivering a substance, such as a treatment agent, to or through a desired area of a blood vessel (a physiological lumen) or tissue in order to treat a localized area of the blood vessel or to treat a localized area of tissue located adjacent to the blood vessel.

Referring to FIG. 2A, delivery assembly 200, in one embodiment, may be in the form of a catheter device that includes delivery lumen 210 that may be formed in a larger catheter body (not shown). The larger catheter body may include one or more lumens to accommodate, for example, a guidewire, an inflation balloon, and/or an imaging device. Further, such a catheter body may accommodate one or more delivery lumens, such as delivery lumen 210. Delivery lumen 210, in this example, extends between distal portion 205 and proximal portion 215 of delivery assembly 200. Delivery lumen 210 can be made from any suitable material, such as polymers and co-polymers of polyamides, polyolefins, polyurethanes, and the like.

In one embodiment, delivery assembly 200 includes needle 220 movably disposed within delivery lumen 210. Needle 220 is, for example, a stainless steel hypotube that extends a length of the delivery assembly. Needle 220 includes a lumen with an inside diameter of, representatively, 0.16 inches (0.40 centimeters). In one example for a retractable needle catheter, needle 220 has a length of about 40 inches (1.6 meters) from distal portion 205 to proximal portion 215. The needle 220 may include at least one opening 230. At an end of proximal portion 215 is adapter 250 of, for example, a female luer housing.

When loaded, a substance may be introduced according to known substance delivery techniques such as by advancing tip 240 of needle 220 into tissue (e.g., a wall of a blood vessel) and delivering the substance through back pressure (e.g., pressure applied at proximal portion 215, such as by a needle luer). In some applications, delivery assembly 200 enters percutaneously through the left ventricle of the heart.

FIG. 2B illustrates an alternative delivery assembly which can be used in conjunction with embodiments of the present invention. In some embodiments, delivery device 260 is a syringe. Delivery device 260 may include a body 270, a needle 280 and a plunger 290. A shaft of plunger 290 has an exterior diameter slightly less than an interior diameter of body 270 so that plunger 290 can, in one position, retain a substance in body 270 and, in another position, push a substance through needle 280. Syringes are known by those skilled in the art. In some applications, delivery device 260 may be applied directly to a treatment site during an open-chest surgery procedure.

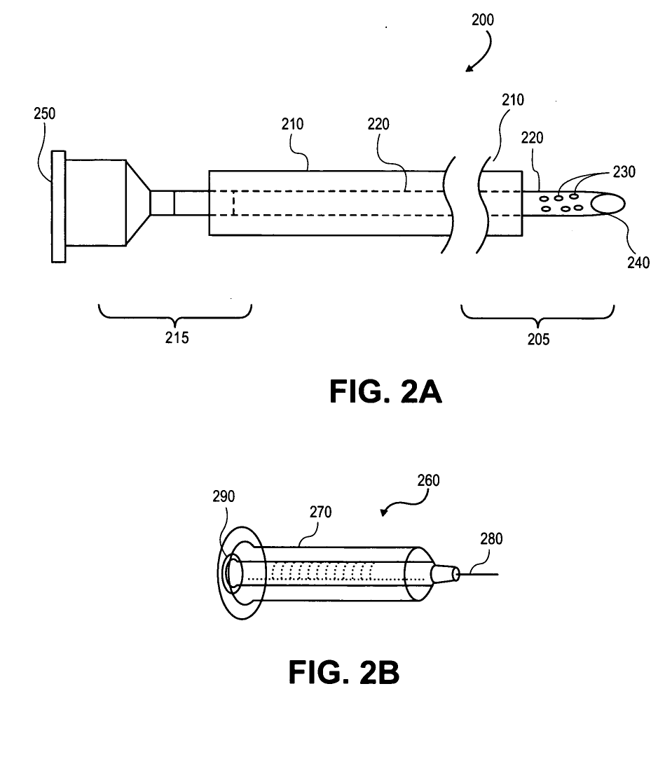

FIG. 3 illustrates a delivery device 300, hereinafter interchangeably referred to as a needle, known in the art. Needle 300 includes a cylindrical hollow body 310, a proximal portion 320 and a distal portion 330. Proximal portion 320 is in fluid communication with a substance reservoir (not shown). Distal portion includes a tip 340, which can be tapered to aid in piercing tissue, with an opening 350. Opening 350 is in fluid communication with a lumen 360 and delivers injectate 370 to a treatment site, or target tissue, in the body. Examples of treatment sites include vessels and organs.

When needle 300 punctures the target tissue, opening 350 can be sealed by the surrounding tissue. The injectate can potentially create damage to the surrounding tissue due to its high focal injection pressure. In addition, since all of the injectate is released into one focal area, the tissue space around opening 350 can lead to backflow into surrounding tissue thereby minimizing the potential benefits to target tissue and decreasing the ability of the injectate to adequately disperse to the target tissue region. As a result, multiple injections are often required to achieve full treatment coverage to the target tissue region.

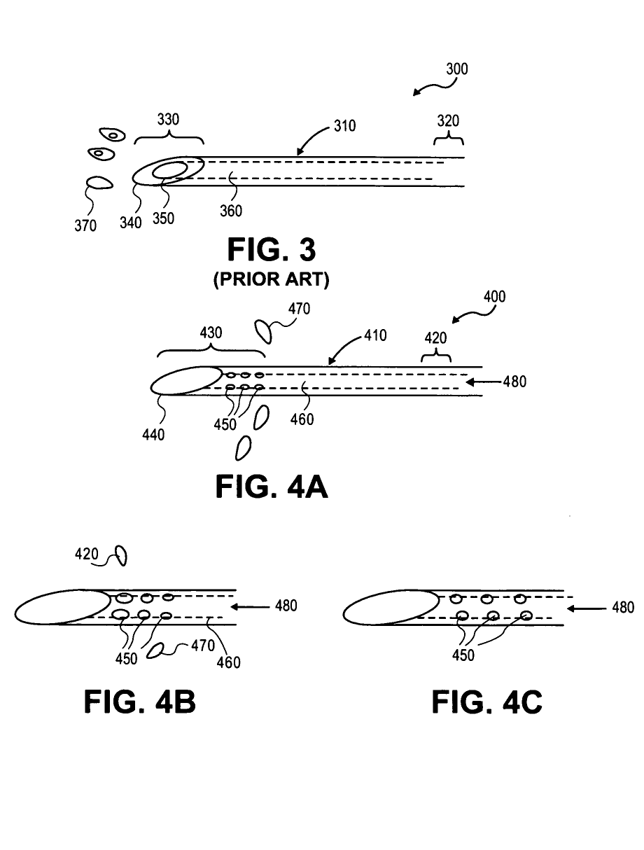

FIG. 4A illustrates one embodiment of a delivery device of the present invention. The delivery device or needle 400 includes a cylindrical hollow body 410 (shaft), a proximal portion 420 and a distal portion 430. The proximal portion 420 is in fluid communication with a substance reservoir (not shown). The distal portion can include a tip 440, which can be tapered, conical or otherwise shaped such that it has the ability to pierce tissue at a target tissue region. In some embodiments, tip 440 can be sealed or, alternatively, have a reduced opening in fluid communication with a lumen 460 (not shown). Distal portion 430 can include multiple circumferential openings 450 which are in fluid communication with lumen 460. Openings 450 can be configured in arrays, such as rows, or any other suitable pattern. Openings 450 may be disposed radially partially or completely around the circumference of body 410.

In some embodiments, openings 450 can have the same, or substantially the same, diameter. The diameter can be in a range from about 0.002 inches to about 0.020 inches. Openings 450 can be aligned in at least one or more rows and spaced evenly in a radial direction. When injectate 470 flows through lumen 460 (arrow 480), the injectate will be expelled through multiple openings 450. The openings closest to the proximal portion 420 of the shaft 410 will have higher flow than the openings closer to the distal portion 430 due to the increase in flow resistance with increasing flow resistance.

In some embodiments, openings 450 can have varying diameters (see FIG. 4B). For example, openings 450 can be aligned in at least two rows and spaced evenly in a radial direction. Openings 450 can increase in diameter from the most proximal openings to the most distal openings. The increase in diameter of openings 450 progressing from the proximal openings to the distal openings can compensate for the resistance resulting from the longer distance injectate 470 has to travel down lumen 460 (arrow 480) to reach the target tissue.

In some embodiments, openings 450 can be staggered in a radial direction (see FIG. 4C). Openings 450 can have the same diameter, varying diameters or a combination of both depending on the arrangement of openings 450 in arrays, rows or other suitable configurations. The staggering of the multiple openings 450 can help maintain shaft strength and integrity.

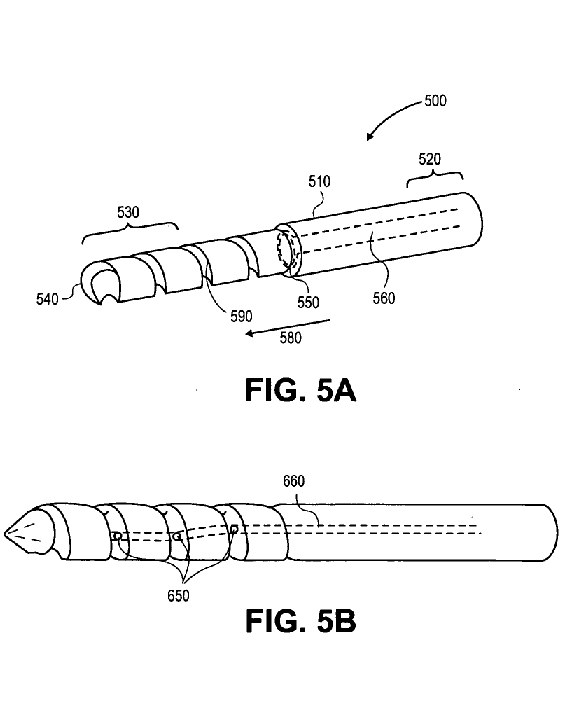

In any of the above-mentioned embodiments, the multiple openings can be recessed relative to the shaft. In some embodiments, a recess can be located at a distal end of a needle. For example, a circumferential groove in a helical configuration may be machined onto a needle tip during the manufacturing process. In some embodiments, the multiple openings may be machined into the circumferential groove. (see FIGS. 5A-5B) The recessing around the multiple openings can allow for more space for the injectate to fill into resulting in reduced backflow. Moreover, the multiple openings can be circular-shaped, oval-shaped or any other suitable configuration.

In some embodiments of the present invention, the tip of a needle can be modified to increase injectate dispersion throughout the target tissue region. FIGS. 5A-5B illustrate alternative embodiments of a delivery device of the present invention. The delivery device or needle 500, includes a hollow cylindrical body 510, a proximal portion 520 and a distal portion 530. Body 510 includes a lumen 560 with an opening 550 for the delivery of injectate to a target tissue region. Distal portion 530 may be modified by at least one etching or groove 590. A tip 540 may be located at the distal portion 530 and can be flat, conical, tapered or any other suitable configuration (see FIG. 5B). The groove 590 may be in fluid communication with the opening 550. In some embodiments, the opening 550 may be located at tip 540 (not shown). The groove 590 may be modified, etched or otherwise configured during the time of manufacturing or post-manufacturing.

When the needle in the before-described embodiment(s) is applied to a target tissue region, injectate may exit through the opening 550 and continue to flow down groove 590 (see FIG. 5A). In this manner, the injectate disperses throughout a greater area of target tissue region to treat a larger treatment area. In some applications, the injectate may exit through multiple openings 650, which are in fluid communication with the lumen 660 (see FIG. 5B). Thus, the injectate may travel within groove 690 and disperse to the target tissue region to treat a larger treatment area.

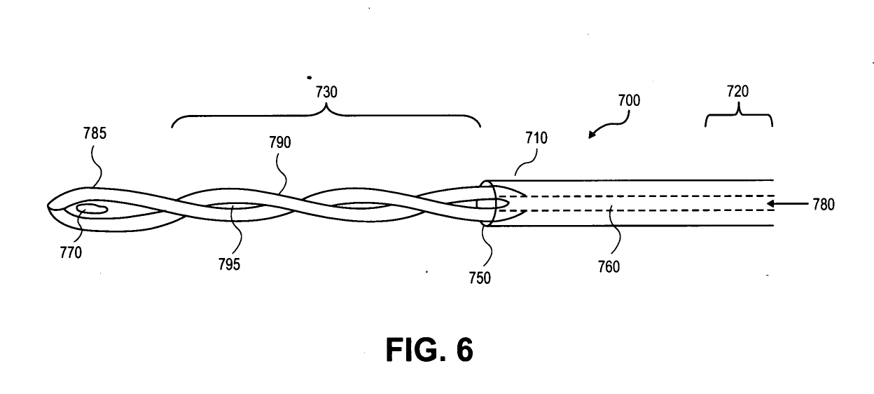

FIG. 6 illustrates an alternative embodiment of a delivery device of the present invention. The delivery device 700 includes a cylindrical hollow body 710 (shaft), a proximal portion 720 and a distal portion 730. The distal portion 720 can include an opening 750 in fluid communication with a lumen 760 with an extending body 790 extending therefrom. Extending body 790 can be any type of helical, spiral or other suitable configuration and can be metal, polymeric, ceramic or a combination thereof. For example, extending body 790 may be at least two extensions twisted together to create a helical extension 785 thereof. Extension extending body 790 should be sturdy enough to withstand pressure when injected into a target tissue area and can be made of, for example, stainless steel. In some embodiments, extending body 790 can be hollow or solid. In some embodiments, shaft 785 can be flexible for penetration into the tissue region in a curved manner.

When applied to a target tissue region, extending body 790 can create a space in which injectate 770 may be dispersed to a larger treatment area. In some embodiments, an interstitial channel 795 is formed within the helical configuration of extending body 790 and injectate 770 may flow therethrough. In some applications, extending body 790 may consist of hollow shafts that release injectate 770 through circumferential openings in the body of the hollow shafts (not shown). After release thereof, injectate 770 may subsequently flow through the interstitial channel 795 for greater dispersion to a target tissue region thereof.

In some embodiments, the injectate may include a treatment agent, a contractility-reducing agent or a combination thereof. A treatment agent can include, but is not limited to, an anti-proliferative, an anti-inflammatory or immune modulating agent, an anti-migratory, an anti-thrombotic or other pro-healing agent or a combination thereof. The anti-proliferative agent can be a natural proteineous agent such as cytotoxin or a synthetic molecule or other substances such as actinomycin D, or derivatives and analogs thereof. (manufactured by Sigma-Aldrich 1001 West Saint Paul Avenue, Milwaukee, Wis. 53233; or COSMEGEN available from Merck) (synonyms of actinomycin C1), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone diproprionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen fenclofenac, fenclorac, fendosal, fenpipalone, fentiazac, flazalone, fluazocort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone sodium glycerate, perfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicyclic acid), salicyclic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g., Adriamycin® from Pharmacia & Upjohn, Peapack, N.J.), and mitomycin (e.g., Mutamycin® from Bristol Myers Squibb Co, Stamford, Conn.). Examples of such antiplatelets, anticoagulants, antifebrin, antithrombins include sodium heparin, low molecular weight heparins, heparinoids, hirudin, argatroban, forskolin, vapiprost, prostacyclin, and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc. Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g., Capoten® and Capozide® from Bristol Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other treatment agents which may be appropriate include alpha-interferon, and genetically engineered epithelial cells.

A contractility-reducing agent may be used to stabilize a dynamic organ during, for example, an injection procedure. Examples of contractility-reducing agents include, but are not limited to, heparin, diltiazam and verapamil. In some embodiments, the treatment agent may be combined with the contractility-reducing agent. The foregoing substances are listed by way of example and are not meant to be limiting. Other treatment agents and contractility-reducing agents which are currently available or that may be developed in the future are equally applicable.

From the foregoing detailed description, it will be evident that there are a number of changes, adaptations and modifications of the present invention which come within the province of those skilled in the part. The scope of the invention includes any combination of the elements from the different species and embodiments disclosed herein, as well as subassemblies, assemblies and methods thereof. However, it is intended that all such variations not departing from the spirit of the invention be considered as within the scope thereof.

What is claimed is:

1. A device comprising:
a hollow cylindrical body having dimensions suitable to be placed within a mammal in connection with a medical procedure, the body comprising a distal portion and a proximal portion, wherein the proximal portion is adapted to couple to a substance delivery device and wherein the distal portion is adapted to expel a substance through at least one circumferential opening in fluid communication with a lumen.

2. The device of claim 1, wherein the body is a needle.

3. The device of claim 2, wherein the distal portion comprises a distal tip.

4. The device of claim 3, wherein the distal tip is a closed tip or an opened tip.

5. The device of claim 1, wherein the at least one circumferential opening is recessed.

6. The device of claim 1, wherein the distal portion comprises multiple circumferential openings.

7. The device of claim 6, wherein the multiple circumferential openings are same-sized openings or different-sized openings.

8. The device of claim 6, wherein the multiple circumferential openings are a staggered array or an aligned array.

9. A device comprising:
a cylindrical body comprising a portion having a lumen therethrough, the body comprising a distal portion and a proximal portion, wherein the proximal portion is adapted to couple to a substance delivery device and wherein the distal portion comprises (a) a circumferential groove and (b) at least one opening in fluid communication with the lumen.

10. The device of claim 9, wherein the body is a needle.

11. The device of claim 9, wherein the body is a shaft.

12. The device of claim 11, wherein the distal portion comprises at least two wires in a helical conformation.

13. The device of claim 10, wherein the groove is in fluid communication with the opening.

14. The device of claim 12, wherein the groove is in fluid communication with the opening.

15. The device of claim 10, wherein the openings comprise multiple circumferential openings.

16. A method of delivering a substance with a needle wherein the fluid is expelled between an interface surface and a point of entry in physiological tissue.

17. The method of claim 16, wherein the interface surface is defined as the surface between a tip of the needle and physiological tissue in contact with the tip of the needle.

18. The method of the claim 16, wherein the substance comprises at least one of a treatment agent and a contractility-reducing agent.

19. A method comprising:
introducing a needle into a treatment site wherein a tip of the needle in contact with physiological tissue defines an interface surface;
after introducing the needle, injecting a substance between the interface surface and a point of entry in physiological tissue.

20. The method of the claim 19, wherein the substance comprises at least one of a treatment agent and a contractility-reducing agent.
